(19) United States
(12) Patent Application Publication
(10) Pub. No.: US 2024/0269090 A1
(43) Pub. Date: Aug. 15, 2024

(54) COENZYME Q10 OILING AGENT, PREPARATION METHOD AND USE THEREOF

(71) Applicants: Xiamen Kingdomway Biotech. Co., Ltd., Fujian (CN); Xiamen Kingdomway Group Company, Fujian (CN)

(72) Inventors: Xuerui Wang, Fujian (CN); Jinhong Liu, Fujian (CN); Murong Lin, Fujian (CN); Wenji Wang, Fujian (CN); Jianhua Lan, Fujian (CN)

(73) Assignees: Xiamen Kingdomway Biotech. Co., Ltd., Fujian (CN); Xiamen Kingdomway Group Company, Fujian (CN)

(21) Appl. No.: 18/333,468

(22) Filed: Jun. 12, 2023

(30) Foreign Application Priority Data
Feb. 14, 2023 (CN) 202310111297.8

(51) Int. Cl. A61K 31/122; A61K 9/107

(52) U.S. Cl. CPC A61K 31/122; A61K 9/1075

(57) ABSTRACT

The present disclosure belongs to the technical field of coenzyme Q10, and relates to a coenzyme Q10 oiling agent, a preparation method and a use thereof. The coenzyme Q10 oiling agent comprises the following components in percentage by mass: 20-40% of coenzyme Q10, 45-65% of mixed oil, 10-15% of biphase emulsifying agent, wherein the coenzyme Q10 in the coenzyme Q10 oiling agent is present in a molecular form above 0° C.; the mixed oil simultaneously contains a saturated fatty acid (ester) and an unsaturated fatty acid (ester); the biphase emulsifying agent simultaneously contains a hydrophilic emulsifying agent and a lipophilic emulsifying agent. The coenzyme Q10 in the coenzyme Q10 oiling agent provided by the present disclosure still can be stably present in the molecular form at high content, and therefore has high bioavailability.

### CROSS-REFERENCE TO RELATED APPLICATION

This application claims the priority benefit of China application serial no. 202310111297.8, filed on Feb. 14, 2023. The entirety of the above-mentioned patent application is hereby incorporated by reference herein and made a part of this specification.

### BACKGROUND

### Technical Field

The present disclosure belongs to the technical field of coenzyme Q10, and relates to a coenzyme Q10 oiling agent, a preparation method therefor and use thereof.

### Description of Related Art

Coenzyme Q10 (CoQ10) is a good biological drug, which is an element that can produce energy in a human body, exists in a cytomembrane of each visceral organ, and is capable of improving myocardial metabolism and strengthening cardiac functions. The coenzyme Q10 also has excellent antioxidant and free radical scavenging functions, and is capable of preventing the peroxidation of lipid in a vascular wall, preventing atherosclerosis and improving body immunity. As a fat-soluble quasi vitamin, the coenzyme Q10 is insoluble in water, which makes it difficult to digest and absorb in the human body. Although it is diluted and dissolved by using the oiling agent, the coenzyme Q10 has no good effect due to its limited dissolubility in oil and a crystalline state. Therefore, it is needed to take excessive coenzyme Q10 to achieve an expected effect.

Currently, most of the coenzyme Q10 soft capsule products available in the market use single plant oil such as soybean oil, corn oil and sunflower seed oil as auxiliary dissolving oiling agents, leading to single efficacy. Moreover, the coenzyme Q10 itself has the characteristics of small dissolubility in plant oil and easy precipitation and crystallization, which restricts the improvement of the content of coenzyme Q10 in the product. In addition, the coenzyme Q10 in the existing coenzyme Q10 soft capsule products is basically present in a crystal state, and therefore it is extremely poor in water solubility, low in bioavailability in a human body, and a large number of coenzyme Q10 needs to be supplemented to achieve the expected effect.

### SUMMARY

The objective of the present disclosure is to provide a new coenzyme Q10 oiling agent as well as a preparation method and use thereof to overcome the defect that the coenzyme Q10 in the existing coenzyme Q10 product is easily precipitated in a crystal state, which leads to the defects of difficult to use with high content and poor bioavailability. The coenzyme Q10 in the coenzyme Q10 oiling agent still can be stably present in a molecular form at high content, and is high in bioavailability.

In the preparation process of wet-process grinding, the common practice in this field is to grind the crystals as small as possible to make the particle size of the crystals fine enough, so as to facilitate the dissolution of the crystals into a solvent. However, the inventor of the present disclosure surprisingly finds during the research that the finer the particle size of the coenzyme Q10 crystals is not the better, and the particle size of the coenzyme Q10 crystals is not positive proportional to the solubility in the oiling agent. When the coenzyme Q10 crystals are ground until their particle size D₅₀ is 50-120 nm, the solubility of the coenzyme Q10 in the solvent is the best; when the particle size is further reduced, the dissolution effect is obviously worsen. By deep and broad research, the inventor of the present disclosure further finds that relative to single oil, the use of a saturated fatty acid (ester) and an unsaturated fatty acid (ester) in combination can promote the disappearance of the coenzyme Q10 crystals under the negative pressure so that the coenzyme Q10 is present in a molecular form, and the mixed oil and the coenzyme Q10 can produce an unexpected synergistic effect in the anti-aging aspect so as to be more conducive to the development of the bioactivity of the coenzyme Q10. Furthermore, since the use of the biphase emulsifying agent can endow the coenzyme Q10 with hydrophilicity and lipophilicity, the inherent characteristics of the coenzyme Q10 that is insoluble in water and slightly soluble in oil can be changed, so that the coenzyme Q10 can be stably dispersed into water and oil systems, thereby further enlarging the application range of the coenzyme Q10 so that the coenzyme Q10 can be applied to various kinds of cosmetics, health care products or drugs. In summary, when the coenzyme Q10 crystals is ground to have a specific particle size range of D₅₀=50-120 nm, and dissolved under the negative pressure under the combined action of the mixed oil and the biphase emulsifying agent, it can be dissolved in the molecular form below a melting temperature, so as to maintain a stable non-crystallization effect, thereby obtaining a coenzyme Q10 oiling agent with a high content, stability and no crystal precipitation, a molecular morphology and high bioavailability. On the basis of this, the present disclosure is completed.

Specifically, the present disclosure provides a coenzyme Q10 oiling agent, the coenzyme Q10 oiling agent comprising following components in percentage by mass: 20-40% of coenzyme Q10, 45-65% of mixed oil, and 10-15% of biphase emulsifying agent, wherein the coenzyme Q10 in the coenzyme Q10 oiling agent is present in a molecular form above 0° C.; the mixed oil simultaneously contains a saturated fatty acid (ester) and an unsaturated fatty acid (ester); the biphase emulsifying agent simultaneously contains a hydrophilic emulsifying agent and a lipophilic emulsifying agent.

The present disclosure further provides a method of preparing the coenzyme Q10 oiling agent, the method comprising: grinding coenzyme Q10 crystals in a presence of an oil mixture I and a biphase emulsifying agent to reduce a particle size D₅₀ of the coenzyme Q10 crystals to 50-120 nm, and then evenly mixing an obtained fine grinding fluid with an oil mixture II under a negative pressure to obtain the coenzyme Q10 oiling agent, wherein both the oil mixture I and the oil mixture II contain the mixed oil.

According to the method of preparing the coenzyme Q10 oiling agent provided by the present disclosure, the coenzyme Q10 crystals disappears due to duplex regulation that the internal relies on the solubilizing capacity of the mixed oil and the interaction between the components, and the external relies on the size effect caused by specific particle size, so that it is stably present in the molecular form, thereby fully exerting biological activity. The coenzyme Q10 in the coenzyme Q10 oiling agent provided by the present disclosure is in the molecular state, its molecular arrangement is disordered, and the lattice energy overcome during the dissolution or dissolution is relatively low, which is conducive to entrance of the body fluid and enhancement of bioavailability. In addition, the molecular state of the pure drug is transient, and the crystals are easy to precipitate out after a period of time. In the present disclosure, the molecular state of the coenzyme Q10 is stabilized from the perspective of formulation and preparation methods, thereby prolonging the crystallization and precipitation.

In addition, the present disclosure further provides a use of the coenzyme Q10 oiling agent in preparing cosmetics, health care products or drugs.

The present disclosure has the beneficial effects:

(1) The coenzyme Q10 oiling agent provided by the present disclosure has high content coenzyme Q10 which is up to 40% and can still be stably maintained under the state of such the high content; the coenzyme Q10 crystals cannot be precipitated out above 0° C. so as to facilitate the digestion and absorption; when in use, the coenzyme Q10 does not need to be molten again, which is extremely conducive to preparation and storage of soft capsules; after the coenzyme Q10 is prepared into soft capsules as a content, the solid crystals cannot be precipitated out due to change in environment to cause difficulties in absorption by human body.

(2) In the coenzyme Q10 oiling agent provided by the present disclosure, the mixed oil has good solubility to coenzyme Q10, the use of coenzyme Q10 and the mixed oil in combination in a certain proportion can create the unexpected synergistic effect, the resulting mixture is more significant in anti-aging effect as compared with single oil, and therefore they are stable in compatibility, has no chemical reaction and no adverse response, and is safe and feasible.

In a preferred embodiment, the method of the grinding comprises: performing first-stage grinding after the coenzyme Q10 crystals are mixed with the oil mixture I to reduce the particle size D₅₀ of the coenzyme Q10 crystals to 0.2-1 m, and then performing second-stage grinding after the initial grinding fluid is mixed with a biphase emulsifying agent to reduce the particle size D₅₀ of the coenzyme Q10 crystals in the initial grinding fluid to 50-120 nm. In one aspect, the moderate and efficient crystal refining effect is achieved by low-temperature grinding in two stages in this method, thereby solving the problem of difficult preparation caused by electrostatic adsorption and a low melting point, furthermore, the required particle size range can be reached within short grinding time, and the grinding time is reduced from conventional single cycle grinding of more than 7 hours to less than 3 hours; in another aspect, the adding time of the emulsifying agent is grasped, i.e., the biphase emulsifying agent is added separately when the particle size D₅₀ of the coenzyme Q10 crystals is reduced to 0.2-1 m, which is beneficial to improve the emulsification and embedding effects. The secondary embedding of the emulsifying agent on coenzyme Q10 can be realized by using the method to further protect the crystal, which can not only avoid the crystallization caused by the collision of materials due to mechanical high-speed movement during the grinding, and help to reduce the coenzyme Q10 crystals to the target particle size, but also can allow the coenzyme Q10 to be stably present in the molecular form for a long time so as to prolong the time of crystallization and precipitation, and further facilitate the exertion of the biological activity.

### DESCRIPTION OF THE EMBODIMENTS

In the present disclosure, a coenzyme Q10 oiling agent contains coenzyme Q10, mixed oil and a biphase emulsifying agent, and can also contain an antioxidant. Based on the total weight of the coenzyme Q10 oiling agent, the content of the coenzyme Q10 is 20-40%, such as 20%, 22%, 25%, 28%, 30%, 32%, 35%, 38%, 40% or any value among them; the content of the mixed oil is 45-65%, such as 45%, 48%, 50%, 52%, 55%, 58%, 60%, 62%, 65% or any value among them; the content of the biphase emulsifying agent is 10-15%, such as 10%, 11%, 12%, 13%, 14%, 15% or any value among them; the content of the antioxidant is 0.1-3%, for example 0.1%, 0.5%, 0.8%, 1%, 1.5%, 2%, 2.5%, 3% or any value among them.

In the present disclosure, the coenzyme Q10 in the coenzyme Q10 oiling agent is present in a molecular form above 0° C., that is, the coenzyme Q10 is completely dissolved into the oiling agent and present in an amorphous form.

In the present disclosure, the mixed oil simultaneously contains a saturated fatty acid (ester) and an unsaturated fatty acid (ester). The term “saturated fatty acid (ester)” refers to a saturated fatty acid and/or a saturated fatty acid ester. The term “unsaturated fatty acid (ester)” refers to an unsaturated fatty acid and/or an unsaturated fatty acid ester. In a preferred embodiment, a mass ratio of the saturated fatty acid (ester) to the unsaturated fatty acid (ester) in the mixed oil is 1:(1-2.5), such as 1:1, 1:1.2, 1:1.5, 1:1.8, 1:2, 1:2.2, 1:2.5 or any value among them. In a preferred embodiment, a mass ratio of the saturated fatty acid (ester) to a monounsaturated fatty acid (ester) to a polyunsaturated fatty acid (ester) in the mixed oil is 1:(0-1.5):(1-1.5). Based on 1 part by weight of saturated fatty acid (ester), the content of the monounsatured fatty acid (ester) is 0-1.5 parts by weight, such as 0, 0.2, 0.5, 0.8, 1, 1.2, 1.5 parts by weight or any value among them; the content of the polyunsaturated fatty acid (ester) is 1-1.5 parts by weight, such as 1, 1.1, 1.2, 1.3, 1.4, 1.5 parts by weight or any value among them. The inventor of the present disclosure finds that when the oil is used as above, it is not only more conducive to promote the dissolution of the coenzyme Q10 crystals to make it be stable present in the molecular form, but also more conducive to exert the biological activity of the coenzyme Q10.

In the present disclosure, the saturated fatty acid (ester) is preferred to saturated fatty acid glyceride, such as at least one of triglyceride palmitate, glyceryl stearate, monocaprylin, tricaprin, caprylic capric triglyceride and glyceryl laurate. The specific examples of the monounsaturated fatty acid (ester) include but are not limited to at least one of oleic acid, myristoleic acid and palmitic oleic acid. The specific examples of the polyunsaturated fatty acid (ester) include but are not limited to at least one of α-linolenic acid, eicosapentaenoic acid, docosahexaenoic acid, conjugated linoleic acid, γ-linolenic acid and arachidonic acid.

In the present disclosure, the biphase emulsifying agent simultaneously contains a hydrophilic emulsifying agent and a lipophilic emulsifying agent. A weight ratio of the hydrophilic emulsifying agent to the lipophilic emulsifying agent is (1.5-4):1, such as 1.5:1, 1.8:1, 2:1, 2.2:1, 2.5:1, 2.8:1, 3:1, 3.2:1, 3.5:1, 3.8:1, 4:1 or any value among them. When the hydrophilic emulsifying agent and the lipophilic emulsifying agent are compounded in the above preferred way, the characteristics of the coenzyme Q10 dissolved into water and oil can be well balanced, which is more conducive to stably dispersing the coenzyme Q10 into oil and water systems. Therefore, the coenzyme Q10 has a wide application prospect.

In the present disclosure, the specific examples of the hydrophilic emulsifying agent include but are not limited to at least one of sucrose ester that has a hydrophile-lipophile balance (HLB) value of 13-16, Tween, polyglycerol fatty acid ester and poloxamer. The Tween can be listed as at least one of Tween 20, Tween 40, Tween 60 and Tween 80. The polyglycerol fatty acid ester can be listed as at least one of polyglycerol ester ML-500, polyglycerol ester CR-200 and polyglycerol ester ML-750. The poloxamer can be listed as at least one of poloxamer 124, poloxamer 168 and poloxamer 188.

In the present disclosure, the specific examples of the lipophilic emulsifying agent include but are not limited to at least one of sucrose ester that has an HLB value of 3-6, phospholipid, Span and monoglyceride. The phospholipid can be listed as at least one of lecithin, soybean phospholipids and hydrogenated lecithin. The Span can be listed as at least one of Span 20, Span 40, Span 60 and Span 80. The monoglyceride can be listed as at least one of monoglyceride stearate, monoglyceride lactate, monoglyceride succinate, monoglyceride citrate, monoglyceride oleate and monoglyceride diacetyl tartrate.

In the present disclosure, the antioxidant can be the existing substance capable of effectively avoiding the oxidation of active ingredients. The specific examples of the antioxidant include but are not limited to at least one of vitamin E, tocopherol, a rosemary extract and a tea polyphenol extract.

The present disclosure provides a method of preparing a coenzyme Q10 oiling agent, the method comprising: grinding coenzyme Q10 crystals in a presence of an oil mixture I and a biphase emulsifying agent to reduce a particle size D₅₀ of the coenzyme Q10 crystals to 50-120 nm, and then evenly mixing an obtained fine grinding fluid with an oil mixture II under a negative pressure to obtain the coenzyme Q10 oiling agent, wherein both the oil mixture I and the oil mixture II are the mixture of the mixed oil and the optional antioxidant.

Since the coenzyme Q10 crystals have a large particle size, a temperature higher than the melting point of the coenzyme Q10 is needed to achieve the dissolution of the molecule, however, the higher treatment temperature is not conducive to protecting the activity of the coenzyme Q10. Furthermore, the coenzyme Q10 is easy to agglomerate at the high temperature due to uneven heating, which is not conducive to improvement of bioavailability. In the present disclosure, the coenzyme Q10 is ground to a particle size range of D₅₀=50-120 nm, and the molecular dissolution can be achieved at a temperature lower than the melting point of the coenzyme Q10 under the negative pressure, so as to not only alleviate the burden of machine and save energy consumption but also decrease the destruction of high temperatures on materials. In addition, the coenzyme Q10 crystals have strong oil absorbing capability and soft crystallization hardness; by using the mixed oil as a dispersing agent, the dissolution and dispersion effect is good to result in a fact that the coenzyme Q10 cannot be agglomerated in the process of grinding, and the dispersing system is relatively stable, and the coenzyme Q10 cannot be closely adhered to the inside of a grinding machine to cause wasted materials and difficult cleaning.

Since the coenzyme Q10 crystals have strong electrostatic adsorptivity, large viscidity, easy adhesion to vessels or surfaces of adjuvants and a low melting point (the melting point of the coenzyme Q10 is 48° C.), a part of the coenzyme Q10 can be easily molten and sticky due to raised temperature of a device if a conventional grinding method is used, so as to cause difficult production and bring many inconveniences. The conventional grinding method difficultly reduces the particle size D₅₀ of the coenzyme Q10 crystals to 50-120 nm. The above problems are perfectly solved by the following grinding in stages in the present disclosure.

In a preferred embodiment, the method of the grinding comprises the following steps:
S1, performing a first-stage grinding after the coenzyme Q10 crystals are mixed with the oil mixture I to reduce the particle size D₅₀ of the coenzyme Q10 crystals to 0.2-1 m, so as to obtain an initial grinding fluid; and
S2, performing a second-stage grinding after the initial grinding fluid is mixed with a biphase emulsifying agent to reduce the particle size D₅₀ of the coenzyme Q10 crystals in the initial grinding fluid to 50-120 nm, so as to obtain the fine grinding fluid.

In a preferred embodiment, the method of preparing the coenzyme Q10 oiling agent comprises the following steps:
S1′, stirring and evenly mixing a saturated fatty acid (ester), an unsaturated fatty acid (ester) and optional antioxidant, and dividing the obtained oil mixture into an oil mixture I and an oil mixture II;
S2′, simply stirring and dispersing coenzyme Q10 crystals and the oil mixture I and then performing first-stage grinding in a ball mill to reduce the particle size D₅₀ of the coenzyme Q10 crystals to 0.2-1 m, so as to obtain initial grinding fluid;
S3′, simply stirring and dispersing the initial grinding fluid and a biphase emulsifying agent and then performing a second-stage grinding in the ball mill to reduce the particle size D₅₀ of the coenzyme Q10 crystals in the initial grinding fluid to 50-120 nm, so as to obtain fine grinding fluid; and
S4′, evenly mixing the fine grinding fluid with the oil mixture II under the negative pressure to obtain the coenzyme Q10 oiling agent.

In a preferred embodiment, the conditions of the first-stage grinding are as follows: working temperature is 0° C.-10° C. (such as 0° C., 2° C., 4° C., 6° C., 8° C., 10° C. or any value among them), 70% of ball size is between 0.7 mm and 0.9 mm (such as 0.7 mm, 0.8 mm, 0.9 mm or any value among them), 30% of ball size is between 0.4 mm and 0.6 mm (such as 0.4 mm, 0.5 mm, 0.6 mm or any value among them), and grinding time is 60-90 min (such as 60 min, 65 min, 70 min, 75 min, 80 min, 85 min, 90 min or any value among them).

In a preferred embodiment, the conditions of the second-stage grinding are as follows: working temperature is 10° C.-20° C. (such as 10° C., 12° C., 14° C., 16° C., 18° C., 20° C. or any value among them), ball size is between 0.1 mm and 0.3 mm (such as 0.1 mm, 0.2 mm, 0.3 mm or any value among them), and grinding time is 30-90 min (such as 30 min, 35 min, 40 min, 45 min, 50 min, 55 min, 60 min, 65 min, 70 min, 75 min, 80 min, 85 min, 90 min or any value among them).

In a preferred embodiment, the conditions of evenly mixing the fine grinding fluid with the oil mixture II under the negative pressure are as follows: temperature is 35° C.-40° C., such as 35° C., 36° C., 37° C., 38° C., 39° C., 40° C. or any value among them; pressure is from −0.07 MPa to −0.1 MPa, such as −0.07 MPa, −0.06 MPa, −0.05 MPa, −0.04 MPa, −0.03 MPa, −0.02 MPa, −0.1 MPa or any value among them; time is 15-25 min, such as 15 min, 18 min, 20 min, 22 min, 25 min or any value among them. In the present disclosure, all the pressures refer to gage pressures.

In a preferred embodiment, an amount ratio of the oil mixture I to the oil mixture II is (65%-95%):(5%-35%), such as 65%:35%, 70%:30%, 75%:25%, 80%:20%, 85%:15%, 90%:10%, 95%:5% or any value among them.

In addition, the present disclosure further provides use of the coenzyme Q10 oiling agent in preparing cosmetics, health care products or drugs.

Next, the present disclosure will be described in detail through examples.

### Example 1 Coenzyme Q10 Oiling Agent and Preparation Method Thereof

(1) Raw materials of a coenzyme Q10 oiling agent in this example are as follows:

| Raw materials | Content (wt %) |
|---|---|
| Coenzyme Q10 crystals | 21 |
| Medium-chain triglyceride | 21 |
| (caprylic capric triglycerride) | |
| Myristoleic acid | 21 |
| Conjugated linoleic acid | 21 |
| Soybean phospholipid | 4 |
| Tween-80 | 2 |
| Polyglycerol ester ML-500 | 4 |
| Polyglycerol ester CR-200 | 4 |
| Vitamin E | 2 |

(2) A preparation method of a coenzyme Q10 oiling agent in this example was as follows:
S1′, medium-chain triglyceride, myristoleic acid, conjugated linoleic acid and vitamin E were stirred and evenly mixed to obtain an oil mixture.
S2′, 65% of oil mixture obtained in step S1′ and coenzyme Q10 crystals were simply stirred and dispersed, and then subjected to first-stage grinding in a ball mill, wherein 70% of ball size was between 0.7 mm and 0.9 mm and 30% of ball size was between 0.4 mm and 0.6 mm, the working temperature was 10° C., the ball milling time was 60 min, so as to obtain the initial grinding fluid in which the particle size D₅₀ of coenzyme Q10 crystals was 0.5 m.
S3′, Soybean phospholipid, Tween-80 and polyglycerol ester were added into the initial grinding fluid obtained in step S2′ and then subjected to second-stage grinding in the ball mill, wherein the ball size was between 0.1 mm and 0.3 mm, the working temperature was 10° C., the ball milling time was 45 min, so as to obtain the fine grinding fluid in which the particle size D₅₀ of coenzyme Q10 crystals was 101 nm.
S4′, the fine grinding fluid and 35% of the remaining oil mixture were stirred for 20 min at 38° C. under the negative pressure of −0.08 MPa to be evenly mixed and subsequently cooled to room temperature, so as to obtain the coenzyme Q10 oiling agent, and then the coenzyme Q10 oiling agent was encapsulated into a capsule.

### Example 2 Coenzyme Q10 Oiling Agent and Preparation Method Thereof

(1) Raw materials of a coenzyme Q10 oiling agent in this example are as follows:

| Raw materials | Content (wt %) |
|---|---|
| Coenzyme Q10 crystals | 31 |
| Medium-chain triglyceride | 26 |
| (caprylic capric triglycerride) | |
| Conjugated linoleic acid | 31 |
| Soybean phospholipid | 4 |
| Tween-80 | 2 |
| Polyglycerol ester ML-750 | 5 |
| Vitamin E | 1 |

(2) A preparation method of a coenzyme Q10 oiling agent in this example was as follows:
S1′, medium-chain triglyceride, conjugated linoleic acid and vitamin E were stirred and evenly mixed to obtain an oil mixture.
S2′, 80% of oil mixture obtained in step S1′ and coenzyme Q10 crystals were simply stirred and dispersed, and then subjected to first-stage grinding in a ball mill, wherein 70% of ball size was between 0.7 mm and 0.9 mm and 30% of ball size was between 0.4 mm and 0.6 mm, the working temperature was 5° C., the ball milling time was 70 min, so as to obtain the initial grinding fluid in which the particle size D₅₀ of coenzyme Q10 crystals was 0.8 m.
S3′, Soybean phospholipid, Tween-80 and polyglycerol ester were added into the initial grinding fluid obtained in step S1′ and then subjected to second-stage grinding in the ball mill, wherein the ball size was between 0.1 mm and 0.3 mm, the working temperature was 10° C., the ball milling time was 55 min, so as to obtain the fine grinding fluid in which the particle size D₅₀ of coenzyme Q10 crystals was 115 nm.
S4′, the fine grinding fluid and 20% of the remaining oil mixture were stirred for 25 min at 37° C. under the negative pressure of −0.08 MPa to be evenly mixed and subsequently cooled to room temperature, so as to obtain the coenzyme Q10 oiling agent, and then the coenzyme Q10 oiling agent was encapsulated into a capsule.

### Example 3 Coenzyme Q10 Oiling Agent and Preparation Method Thereof

(1) Raw materials of a coenzyme Q10 oiling agent in this example are as follows:

| Raw materials | Content (wt %) |
|---|---|
| Coenzyme Q10 crystals | 31 |
| Medium-chain triglyceride | 10 |
| (caprylic capric triglycerride) | |
| Glyceryl laurate | 6 |
| Palmitic oleic acid | 16 |
| Docosahexaenoic acid (DHA) | 8 |
| Conjugated linoleic acid | 16 |
| Sucrose ester (HLB = 3-6) | 3 |
| Sucrose ester (HLB = 13-16) | 5 |
| Poloxamer 168 | 4 |
| Vitamin E | 1 |

(2) A preparation method of a coenzyme Q10 oiling agent in this example was as follows:
S1′, medium-chain triglyceride, glyceryl laurate, DHA, palmitic oleic acid, conjugated linoleic acid and vitamin E were stirred and evenly mixed to obtain an oil mixture.
S2′, 85% of oil mixture obtained in step S1′ and coenzyme Q10 crystals were simply stirred and dispersed, then subjected to first-stage grinding in a ball mill, wherein 70% of ball size was between 0.7 mm and 0.9 mm and 30% of ball size was between 0.4 mm and 0.6 mm, the working temperature was 5° C., the ball milling time was 65 min, so as to obtain the initial grinding fluid in which the particle size D₅₀ of coenzyme Q10 crystals was 1.0 m.
S3′, sucrose ester (HLB=3-6), sucrose ester (HLB=13-16) and poloxamer 168 were added into the initial grinding fluid obtained in step S2′ and then subjected to second-stage grinding in the ball mill, wherein the ball size was between 0.1 mm and 0.3 mm, the working temperature was 10° C., the ball milling time was 55 min, so as to obtain the fine grinding fluid in which the particle size D₅₀ of coenzyme Q10 crystals was 120 nm.
S4′, the fine grinding fluid and 15% of the remaining oil mixture were stirred for 20 min at 40° C. under the negative pressure of −0.1 MPa to be evenly mixed and then cooled to room temperature, so as to obtain the coenzyme Q10 oiling agent, and then the coenzyme Q10 oiling agent was encapsulated into a capsule.

### Example 4 Coenzyme Q10 Oiling Agent and Preparation Method Thereof

(1) Raw materials of a coenzyme Q10 oiling agent in this example are as follows:

| Raw materials | Content (wt %) |
|---|---|
| Coenzyme Q10 crystals | 40 |
| Triglyceride palmitate | 16 |
| Oleic acid | 8 |
| α-linolenic acid | 4 |
| Conjugated linoleic acid | 20 |
| Span-80 | 2 |
| Tween-80 | 2 |
| Polyglycerol ester ML-750 | 6 |
| Vitamin E | 2 |

(2) A preparation method of a coenzyme Q10 oiling agent in this example was as follows:
S1′, triglyceride palmitate, oleic acid, α-linolenic acid, conjugated linoleic acid and vitamin E were stirred and evenly mixed to obtain an oil mixture.
S2′, 95% of oil mixture obtained in step S1′ and coenzyme Q10 crystals were simply stirred and dispersed, and then subjected to first-stage grinding in a ball mill, wherein 70% of ball size was between 0.7 mm and 0.9 mm and 30% of ball size was between 0.4 mm and 0.6 mm, the working temperature was 5° C., the ball milling time was 85 min, so as to obtain the initial grinding fluid in which the particle size D₅₀ of coenzyme Q10 crystals was 0.6 m.
S3′, Span-80, Tween-80 and polyglycerol ester ML-750 were added into the initial grinding fluid obtained in step S2′ and then subjected to second-stage grinding in the ball mill, wherein the ball size was between 0.1 mm and 0.3 mm, the working temperature was 15° C., the ball milling time was 55 min, so as to obtain the fine grinding fluid in which the particle size D₅₀ of coenzyme Q10 crystals was 76 nm.
S4′, the fine grinding fluid and 5% of the remaining oil mixture were stirred for 25 min at 40° C. under the negative pressure of −0.07 MPa to be evenly mixed and then cooled to room temperature, so as to obtain the coenzyme Q10 oiling agent, and then the coenzyme Q10 oiling agent was encapsulated into a capsule.

### Comparative Example 1

(1) The formulation was the same as that in example 1.

(2) A preparation method of a coenzyme Q10 oiling agent:

The coenzyme Q10 oiling agent was prepared according to the method in example 1 except that in step S4′, the fine grinding fluid was mixed with the oil mixture at room temperature (25° C.) under normal pressure, other conditions were the same as those in example 1, the obtained coenzyme Q10 oiling agent was orange red and opaque, and the coenzyme Q10 in the coenzyme Q10 oiling agent was present in a form of crystal.

### Comparative Example 2

(1) The formulation was the same as that in example 1

(2) A preparation method of a coenzyme Q10 oiling agent:
S1′, medium-chain triglyceride, myristoleic acid, conjugated linoleic acid and vitamin E were stirred and evenly mixed to obtain an oil mixture.
S2′, 65% of oil mixture obtained in step S1′, coenzyme Q10 crystals, soybean lecithin, Tween-80 and polyglycerol ester were simply stirred and dispersed and then added into a ball mill, wherein the ball size was between 0.1 mm and 0.3 mm, the working temperature was 10° C., the ball milling time was 105 min, so as to obtain the grinding fluid in which the particle size D₅₀ of coenzyme Q10 crystals was 2375 m.
S3′, the grinding fluid and 35% of the remaining oil mixture were stirred for 20 min at 38° C. under the negative pressure of −0.08 MPa to be evenly mixed, then cooled to room temperature to obtain the orange red and opaque coenzyme Q10 oiling agent, and the coenzyme Q10 in the coenzyme Q10 oiling agent was still present in a form of crystal.

### Comparative Example 3

(1) The formulation was the same as that in example 1

(2) A preparation method of a coenzyme Q10 oiling agent:
S1′, medium-chain triglyceride, myristoleic acid, conjugated linoleic acid and vitamin E were stirred and evenly mixed to obtain an oil mixture.
S2′, 65% of oil mixture obtained in step S1′, coenzyme Q10 crystals, soybean lecithin, Tween-80 and polyglycerol ester were simply stirred and dispersed, and then subjected to first-stage ball milling in a ball mill, wherein 70% of ball size was between 0.7 mm and 0.9 mm and 30% of ball size is between 0.4 mm and 0.6 mm, the working temperature was 10° C., the ball milling time was 60 min, so as to obtain the initial grinding fluid in which the particle size D₅₀ of coenzyme Q10 crystals was 1.5 m.
S3′, the initial grinding fluid obtained in step S2′ was directly subjected to second-stage grinding, wherein the ball size was between 0.1 mm and 0.3 mm, the working temperature was 10° C., the ball milling time was 45 min, so as to obtain the fine grinding fluid in which the particle size D₅₀ of coenzyme Q10 crystals was 253 m.
S4′, the fine grinding fluid and 35% of the remaining oil mixture were stirred for 20 min at 38° C. under the negative pressure of −0.08 MPa to be evenly mixed, and then cooled to room temperature to obtain an orange red, transparent and homogenous coenzyme Q10 oiling agent.

### Comparative Example 4

(1) The formulation was the same as that in example 1.

(2) A preparation method of a coenzyme Q10 oiling agent:
the coenzyme Q10 oiling agent was prepared according to the method in example 1 except that the ball milling time in step S3′ was changed into 370 min, the obtained fine grinding fluid had a coenzyme Q10 crystals particle size D₅₀ of 45 nm, and other conditions were the same as those in example 1, so as to obtain an orange red, transparent and homogenous coenzyme Q10 oiling agent.

### Comparative Example 5

(1) The formulation was the same as that in example 1.

(2) A preparation method of a coenzyme Q10 oiling agent:
the coenzyme Q10 oiling agent was prepared according to the method in example 1 except that the ball milling time in step S3′ was changed into 780 min, the obtained fine grinding fluid had a coenzyme Q10 crystals particle size D₅₀ of 13 nm, and other conditions were the same as those in example 1, so as to obtain an orange red, transparent and homogenous coenzyme Q10 oiling agent.

### Comparative Example 6

(1) The formulation was the same as that in example 1

(2) A preparation method of a coenzyme Q10 oiling agent:
the coenzyme Q10 oiling agent was prepared according to the method in example 1 except that the ball milling time in step S3′ was changed into 20 min, the obtained fine grinding fluid had a coenzyme Q10 crystals particle size D₅₀ of 300 nm, and other conditions were the same as those in example 1, so as to obtain an orange red, transparent and homogenous coenzyme Q10 oiling agent.

### Comparative Example 7

(1) Myristoleic acid and conjugated linoleic acid were replaced with medium-chain triglyceride in equal weight parts, and other components were the same as the formulation in example 1.

(2) A preparation method of a coenzyme Q10 oiling agent:
the coenzyme Q10 oiling agent was prepared according to the method in example 1 except that in step S1′, myristoleic acid and conjugated linoleic acid were replaced with medium-chain triglyceride, and other conditions were the same as those in example 1, so as to obtain an orange red and opaque coenzyme Q10 oiling agent.

### Comparative Example 8

(1) Medium-chain triglyceride was replaced with myristoleic acid in equal weight parts, and other components were the same as the formulation in example 1.

(2) A preparation method of a coenzyme Q10 oiling agent:
the coenzyme Q10 oiling agent was prepared according to the method in example 1 except that in step S1′, medium-chain triglyceride was replaced with myristoleic acid in equal weight parts, and other components were the same as the formulation in example 1, so as to obtain an orange red and opaque coenzyme Q10 oiling agent.

### Comparative Example 9

(1) Tween-80, polyglycerol ester ML-500 and polyglycerol ester CR-200 were replaced with soybean lecithin in equal weight parts, and other components were the same as the formulation in example 1.

(2) A preparation method of a coenzyme Q10 oiling agent:
the coenzyme Q10 oiling agent was prepared according to the method in example 1 except that in step S3′, Tween-80, polyglycerol ester ML-500 and polyglycerol ester CR-200 were all replaced with soybean lecithin in equal weight parts, and other components were the same as those in example 1, so as to obtain an orange red and opaque coenzyme Q10 oiling agent.

### Comparative Example 10

(1) Soyabean lecithin was replaced with Tween-80 in equal weight parts, and other components were the same as the formulation in example 1.

(2) A preparation method of a coenzyme Q10 oiling agent:
the coenzyme Q10 oiling agent was prepared according to the method in example 1 except that in step S3′, soybean lecithin was replaced with Tween-80 in equal weight parts, and other conditions were the same as those in example 1, so as to obtain an orange red and opaque coenzyme Q10 oiling agent.

### Test Example 1 Thermostability of Coenzyme Q10 Oiling Agent

The thermostability of oiling agents prepared in examples 1-4 and comparative examples 1-10 was tested to observe whether the oil agent was separated, turbid and precipitated at different temperatures. The oiling agents prepared in examples 1-4 and comparative examples 1-10 were stored in thermostats at 0° C., 25° C. and 45° C. After one week, samples were visually observed to determine the stability of the oiling agent system (when the coenzyme Q10 was in a molecular form, the oiling agent system was clear, transparent and homogenous; when coenzyme Q10 was crystallized and precipitated, the oiling agent system was separated, turbid or precipitated). The results are shown in Table 1 below.

TABLE 1

| | | Observed after placement one week | | |
|---|---|---|---|---|
| Sample | Initial state | 0° C. | 25° C. | 45° C. |
| Example 1 | Orange red, | No obvious | No obvious | Slight dark, a system |
| | transparent and | change | change | has no obvious change |
| | homogenous | | | |
| | oiling agent | | | |
| Example 2 | Orange red, | No obvious | No obvious | Slight dark, a system |
| | transparent and | change | change | has no obvious change |
| | homogenous | | | |
| | oiling agent | | | |
| Example 3 | Orange red, | No obvious | No obvious | Slight dark, a system |
| | transparent and | change | change | has no obvious change |
| | homogenous | | | |
| | oiling agent | | | |
| Example 4 | Orange red, | No obvious | No obvious | slight dark, a system |
| | transparent and | change | change | has no obvious change |
| | homogenous | | | |
| | oiling agent | | | |
| Comparative | Orange red | Crystal | Crystal | Deepened color, crystal |
| example 1 | oiling agent, | precipitated | precipitated | precipitated |
| | opaque | | | |
| Comparative | Orange red | Crystal | Crystal | Deepened color, crystal |
| example 2 | oiling agent, | precipitated | precipitated | precipitated |
| | opaque | | | |
| Comparative | Orange red, | No obvious | Crystal | slight dark, crystal |
| example 3 | transparent and | change | precipitated | precipitated |
| | homogenous | | | |
| | oiling agent | | | |
| Comparative | Orange red, | No obvious | No obvious | slight dark, crystal |
| example 4 | transparent and | change | change | precipitated |
| | homogenous | | | |
| | oiling agent | | | |
| Comparative | Orange red, | No obvious | No obvious | slight dark, crystal |
| example 5 | transparent and | change | change | precipitated |
| | homogenous | | | |
| | oiling agent | | | |
| Comparative | Orange red, | No obvious | Crystal | Deepened color, Crystal |
| example 6 | transparent and | change | precipitated | precipitated |
| | homogenous | | | |
| | oiling agent | | | |
| Comparative | Orange red | Crystal | Crystal | Deepened color, crystal |
| example 7 | oiling agent, | precipitated | precipitated | precipitated |
| | opaque | | | |
| Comparative | Orange red | Crystal | Crystal | Deepened color, crystal |
| example 8 | oiling agent, | precipitated | precipitated | precipitated |
| | opaque | | | |
| Comparative | Orange red | Crystal | Crystal | Deepened color, crystal |
| example 9 | oiling agent, | precipitated | precipitated | precipitated |
| | opaque | | | |
| Comparative | Orange red | Crystal | Crystal | Deepened color, crystal |
| example 10 | oiling agent, | precipitated | precipitated | precipitated |
| | opaque | | | |

It can be seen from Table 1 that the coenzyme Q10 oiling agents provided by the present disclosure can all be kept stable within a wide temperature range, the good transparency of the oiling agent systems is kept, and there are no phase separation, turbidity and crystal precipitation in the oiling agent systems.

### Test Example 2 Anti-Aging Performance of Coenzyme Q10 Oiling Agent

Sample 1: a coenzyme Q10 oiling agent prepared in example 1.

Sample 2: a coenzyme Q10 oiling agent prepared in example 2.

Sample 3: a coenzyme Q10 oiling agent prepared in example 3.

Sample 4: a coenzyme Q10 oiling agent prepared in example 4.

Reference sample 1: the coenzyme Q10 in the formulation in example 1 was replaced with soybean oil in equal weight, other components and conditions were consistent with those in example 1, and the obtained oiling agent was used as reference sample 1.

Reference sample 2: the mixed oil in the formulation in example 1 was replaced with soybean oil in equal weight, other components and conditions were consistent with those in example 1, and the obtained oiling agent was used as reference sample 2.

Reference sample 3: a coenzyme Q10 oiling agent prepared in comparative example 1.

Reference sample 4: a coenzyme Q10 oiling agent prepared in comparative example 2.

Reference sample 5: a coenzyme Q10 oiling agent prepared in comparative example 3.

Reference sample 6: a coenzyme Q10 oiling agent prepared in comparative example 4.

Reference sample 7: a coenzyme Q10 oiling agent prepared in comparative example 5.

Reference sample 8: a coenzyme Q10 oiling agent prepared in comparative example 6.

Reference sample 9: a coenzyme Q10 oiling agent prepared in comparative example 7.

Reference sample 10: a coenzyme Q10 oiling agent prepared in comparative example 8.

Reference sample 11: a coenzyme Q10 oiling agent prepared in comparative example 9.

Reference sample 12: a coenzyme Q10 oiling agent prepared in comparative example 10.

The above 16 samples were subjected to animal experiment.

Modeling: 180 Wistar rats were taken with weights of 120.45 g±3.12 g, half male and half female, and randomly divided into 18 groups for 10 rats in each group. In addition to blank control group, rats in remained groups were subcutaneously injected with 120 mg/kg 5% D-galactose every day to establish an aging model.

Administration: normal saline was added into oiling agents in sample 1, sample 2, sample 3, sample 4, reference sample 1, reference sample 2, reference sample 3, reference sample 4, reference sample 5, reference sample 6, reference sample 7, reference sample 8, reference sample 9, reference sample 10, reference sample 11 and reference sample 12 respectively to prepare 2.0 g/mL suspensions (2 g of oiling agent required 1 mL normal saline). Starting from the day 11 of modeling, continuous intragastric administration was performed for 30 days, and the administration volume in each group was 2 mL. Equal volume of normal saline was administrated to blank control group and model control group. After administration each time, the rats were fasted but allowed to drink water for 24 h, the blood was fetched from eye grounds and underwent standing at room temperature until being completed clotted and the centrifuged to isolate serum for later use. The SOD activity, GSH-Px activity and MDA content of the serum were measured by using a xanthine oxidase method, a dithiodinitrobenzoic acid method and a thiobarbituric acid spectrophotometry.

(1) The effect of samples on MDA content of serum: mice were continuously gavaged with test samples for 30 days via oral administration. The detection results of the MDA content of lipid peroxide in the serum are seen in Table 2.

TABLE 2

| | Number of | MDA content | Decrease (%, VS |
|---|---|---|---|
| Group | animals | (nmol/mL) | Model control group) |
| Blank control group | 10 | 4.15 ± 0.85 | 47.9 |
| Model control group | 10 | 7.97 ± 2.67 | — |
| Sample group 1 | 10 | 4.61 ± 0.75 | 42.1 |
| Sample group 2 | 10 | 4.18 ± 0.82 | 47.6 |
| Sample group 3 | 10 | 4.13 ± 0.94 | 48.2 |
| Sample group 4 | 10 | 4.01 ± 0.78 | 49.7 |
| Reference group 1 | 10 | 7.59 ± 2.03 | 4.7 |
| Reference group 2 | 10 | 5.56 ± 0.99 | 30.2 |
| Reference group 3 | 10 | 5.73 ± 1.55 | 28.1 |
| Reference group 4 | 10 | 5.84 ± 1.41 | 26.7 |
| Reference group 5 | 10 | 5.41 ± 1.31 | 32.1 |
| Reference group 6 | 10 | 5.19 ± 1.20 | 34.9 |
| Reference group 7 | 10 | 5.15 ± 1.08 | 35.4 |
| Reference group 8 | 10 | 5.27 ± 1.25 | 33.8 |
| Reference group 9 | 10 | 5.53 ± 1.13 | 30.6 |
| Reference group 10 | 10 | 5.54 ± 1.22 | 30.4 |
| Reference group 11 | 10 | 5.46 ± 1.38 | 31.5 |
| Reference group 12 | 10 | 5.49 ± 1.43 | 31.1 |

(2) The effect of samples on SOD activity of serums of mice: mice were continuously gavaged with test samples for 30 days via oral administration. The detection results of the SOD activity of the serum are seen in Table 3.

TABLE 3

| | Number of | SOD | Increase (%, VS |
|---|---|---|---|
| Group | animals | (U/mL) | model control group) |
| Blank control group | 10 | 176.7 ± 16.1 | 69.4 |
| Model control group | 10 | 104.3 ± 19.3 | — |
| Sample group 1 | 10 | 170.1 ± 16.4 | 63.1 |
| Sample group 2 | 10 | 176.2 ± 15.9 | 68.9 |
| Sample group 3 | 10 | 176.9 ± 17.3 | 69.6 |
| Sample group 4 | 10 | 182.4 ± 12.8 | 74.8 |
| Reference group 1 | 10 | 115.4 ± 16.8 | 10.6 |
| Reference group 2 | 10 | 152.1 ± 11.7 | 45.8 |
| Reference group 3 | 10 | 151.9 ± 17.2 | 45.6 |
| Reference group 4 | 10 | 149.5 ± 13.3 | 43.3 |
| Reference group 5 | 10 | 156.7 ± 14.8 | 50.2 |
| Reference group 6 | 10 | 163.4 ± 13.6 | 56.7 |
| Reference group 7 | 10 | 164.7 ± 15.1 | 57.9 |
| Reference group 8 | 10 | 162.9 ± 13.9 | 56.2 |
| Reference group 9 | 10 | 152.8 ± 14.3 | 46.5 |
| Reference group 10 | 10 | 152.4 ± 15.5 | 46.1 |
| Reference group 11 | 10 | 153.6 ± 14.6 | 47.3 |
| Reference group 12 | 10 | 154.1 ± 13.8 | 47.7 |

(3) The effect of samples on GHS-PX activity of serums of mice: mice were continuously gavaged with test samples for 30 days via oral administration. The detection results of the GHS-PX activity of the serum are seen in Table 5.

TABLE 5

| | Number of | GSH-PX | Increase (%, VS |
|---|---|---|---|
| Group | animals | (U/mL) | model control group) |
| Blank control group | 10 | 86.4 ± 3.4 | 62.4 |
| Model control group | 10 | 53.2 ± 4.8 | — |
| Sample group 1 | 10 | 84.6 ± 2.7 | 59.0 |
| Sample group 2 | 10 | 86.5 ± 2.5 | 62.6 |
| Sample group 3 | 10 | 87.1 ± 2.3 | 63.7 |
| Sample group 4 | 10 | 89.6 ± 2.1 | 68.4 |
| Reference group 1 | 10 | 57.4 ± 3.7 | 7.9 |
| Reference group 2 | 10 | 74.8 ± 4.8 | 40.6 |
| Reference group 3 | 10 | 74.0 ± 3.1 | 39.1 |
| Reference group 4 | 10 | 73.3 ± 3.9 | 37.8 |
| Reference group 5 | 10 | 77.6 ± 4.2 | 45.9 |
| Reference group 6 | 10 | 79.2 ± 3.4 | 48.9 |
| Reference group 7 | 10 | 79.9 ± 3.5 | 50.2 |
| Reference group 8 | 10 | 79.0 ± 3.8 | 48.5 |
| Reference group 9 | 10 | 75.5 ± 3.2 | 41.9 |
| Reference group 10 | 10 | 75.1 ± 3.7 | 41.2 |
| Reference group 11 | 10 | 75.9 ± 4.0 | 42.6 |
| Reference group 12 | 10 | 76.2 ± 4.5 | 43.2 |

In this experiment, the serum MDA of the model control group was always higher than that of blank control group, and was statistically significant, indicating that peroxidation damage mode was successful, and the results were credible. After mice in sample group 1, sample group 2, sample group 3, sample group 4, reference group 1, reference group 2, reference group 3, reference group 4, reference group 5, reference group 6, reference group 7, reference group 8, reference group 9, reference group 10, reference group 11 and reference group 12 were continuously gavaged for 30 days, the MDA content of peroxide lipid degraded product in animal serum was significantly decreased compared with that of model control group; the SOD levels of the serums in sample group 1, sample group 2, sample group 3 and sample group 4 were significantly increased compared with those in model control groups and superior to those in reference groups; the GSH-PX levels of the serums of animals in sample group 1, sample group 2, sample group 3 and sample group 4 were significantly increased compared with those in model control groups and superior to those in reference groups. The results showed that sample 1, sample 2, sample 3 and sample 4 had excellent anti-aging functions. It can be seen that the coenzyme Q10 oiling agent provided by the present disclosure can effectively enhance the body's ability to resist oxidation and remove free radicals, reduce the damage degree of tissue cells, and basically restore the indexes to the level of non-aging modeling. It can be seen from result comparison of sample group 1 versus reference group 4, reference group 5, reference group 6, reference group 7 and reference group 8 that the coenzyme Q10 crystals are ground into a specific particle size range of D₅₀=50-120 nm, and the obtained coenzyme Q10 oiling agent has more excellent anti-aging function under the combined action of the mixed oil and the biphase emulsifying agent. It can be seen that result comparison of sample group 1 versus reference group 1, reference group 2, reference group 9, reference group 10, reference group 11, and reference group 12 that the coenzyme Q10 in combination with the mixed oil and the bipolar emulsifying agents can exert unexpected synergic effect in the aspect of anti-aging function.

The coenzyme Q10 oiling agent provided by the present disclosure is in the molecular state, its molecular arrangement is disordered, and the lattice energy overcome during the dissolution or dissolution is relatively low, which is conducive to entrance of the body fluid and enhancement of bioavailability. In addition, the molecular state of the simple drug is transient, and the crystals are easy to precipitate out after a period of time. In the present disclosure, the molecular state of the coenzyme Q10 is stabilized from the perspective of formulation and preparation methods, thereby prolonging the crystallization and precipitation.

Although the embodiments of the present disclosure have been shown and described above. It should be understood that the above embodiments are only exemplary, and cannot be understood as limiting the present disclosure. Changes, modifications, replacements and variations can be made by a person of ordinary skill in the art without departing from the principle and spirit of the present disclosure.

1. A coenzyme Q10 oiling agent, the coenzyme Q10 oiling agent comprising following components in percentage by mass:
20-40% of coenzyme Q10, 45-65% of mixed oil, and 10-15% of biphase emulsifying agent,
wherein the coenzyme Q10 in the coenzyme Q10 oiling agent is present in a molecular form above 0° C.;
the mixed oil simultaneously contains a saturated fatty acid (ester) and an unsaturated fatty acid (ester);
the biphase emulsifying agent simultaneously contains a hydrophilic emulsifying agent and a lipophilic emulsifying agent.

2. The coenzyme Q10 oiling agent according to claim 1, wherein a mass ratio of the saturated fatty acid (ester) to the unsaturated fatty acid (ester) in the mixed oil is 1:(1-2.5).

3. The coenzyme Q10 oiling agent according to claim 2, wherein a mass ratio of the saturated fatty acid (ester) to a monounsaturated fatty acid (ester) to a polyunsaturated fatty acid (ester) in the mixed oil is 1:(0-1.5):(1-1.5).

4. The coenzyme Q10 oiling agent according to claim 2, wherein the saturated fatty acid (ester) is saturated fatty acid glyceride.

5. The coenzyme Q10 oiling agent according to claim 3, wherein the monounsaturated fatty acid (ester) is selected from at least one of oleic acid, myristoleic acid and palmitic oleic acid.

6. The coenzyme Q10 oiling agent according to claim 3, wherein the polyunsaturated fatty acid (ester) is selected from at least one of α-linolenic acid, eicosapentaenoic acid, docosahexaenoic acid, conjugated linoleic acid, γ-linolenic acid and arachidonic acid.

7. The coenzyme Q10 oiling agent according to claim 1, wherein a weight ratio of the hydrophilic emulsifying agent to the lipophilic emulsifying agent is (1.5-4):1; the hydrophilic emulsifying agent is selected from at least one of sucrose ester that has an HLB value of 13-16, Tween, polyglycerol fatty acid ester and poloxamer; the lipophilic emulsifying agent is selected from at least one of sucrose ester that has an HLB value of 3-6, phospholipid, Span and monoglyceride.

8. The coenzyme Q10 oiling agent according to claim 1, wherein the coenzyme Q10 oiling agent also contains an antioxidant with a mass percentage of 0.1-3%.

9. The coenzyme Q10 oiling agent according to claim 8, wherein the antioxidant is selected from at least one of vitamin E, tocopherol, a rosemary extract and a tea polyphenol extract.

10. A method of preparing the coenzyme Q10 oiling agent according to claim 1, comprising:
grinding coenzyme Q10 crystals in a presence of an oil mixture I and a biphase emulsifying agent to reduce a particle size D₅₀ of the coenzyme Q10 crystals to 50-120 nm, and then evenly mixing an obtained fine grinding fluid with an oil mixture II under a negative pressure to obtain the coenzyme Q10 oiling agent,
wherein both the oil mixture I and the oil mixture II contain the mixed oil.

11. The method according to claim 10, a method of the grinding comprises following steps:
S1, performing a first-stage grinding after the coenzyme Q10 crystals are mixed with the oil mixture I to reduce the particle size D₅₀ of the coenzyme Q10 crystals to 0.2-1 μm, so as to obtain an initial grinding fluid; and
S2, performing a second-stage grinding after the initial grinding fluid is mixed with the biphase emulsifying agent to reduce the particle size D₅₀ of the coenzyme Q10 crystals in the initial grinding fluid to 50-120 nm, so as to obtain the fine grinding fluid.

12. The method according to claim 11, wherein conditions of the first-stage grinding are as follows:
working temperature is 0° C.-10° C., 70% of ball size is between 0.7 mm and 0.9 mm and 30% of ball size is between 0.4 mm and 0.6 mm, and grinding time is 60-90 min;
conditions of the second-stage grinding are as follows:
working temperature is 10° C.-20° C., ball size is between 0.1 mm and 0.3 mm, and grinding time is 30-90 min.

13. The method according to claim 10, wherein conditions of evenly mixing the fine grinding fluid with the oil mixture II under the negative pressure are as follows:
temperature is 35° C.-40° C., pressure is from −0.07 MPa to −0.1 MPa, and time is 15-25 min.

14. The method according to claim 10, wherein an amount ratio of the oil mixture I to the oil mixture II is (65%-95%):(5%-35%).

15. A use of the coenzyme Q10 oiling agent according to claim 1 in preparing cosmetics, health care products or drugs.
